(19) United States
(12) Patent Application Publication
(10) Pub. No.: US 2010/0240868 A1
(43) Pub. Date: Sep. 23, 2010

(54) Composition and Method for a Producing Stable Amyloid Beta Oligomers of High Molecular Weight

(76) Inventors: Henryk Mach, Ambler, PA (US); Denise Nawrocki, West Point, PA (US); David Thiriot, Harleysville, PA (US); Robert Evans, Souderton, PA (US)

Correspondence Address: MERCK, P O BOX 2000, RAHWAY, NJ, 07065-0907, US

(21) Appl. No.: 11/922,528

(22) PCT Filed: Jun. 26, 2006
(86) PCT No.: PCT/US2006/024744

§ 371 Date: Dec. 19, 2007

Related U.S. Application Data
(60) Provisional application No. 60/695,526, filed on Jun. 30, 2005.

(51) Int. Cl. C07K 14/00

(52) U.S. Cl. 530/350

(57) ABSTRACT

The invention relates to a method for the preparation of a stable, soluble amyloid beta (Aβ) oligomer and composition thereof for use as an antigen for the generation of antibodies for the treatment of Alzheimer's disease and other conditions related to abnormal amyloid beta aggregation. The method which uses a pH in excess of 7.0 and high concentrations of Aβ, optionally includes the use of divalent anions or a helix-inducing solvent to form the oligomers. The stable, soluble Aβ oligomers produced by the method herein have a particle size of 10 to 100 nm in diameter, when measured by a dynamic light scattering technique, and a molecular weight of 100 to 500 kDa.

### FIELD OF THE INVENTION

The invention relates to a method for the preparation of a stable amyloid beta oligomer and composition thereof for use as an antigen or screening reagent for the generation of antibodies for the treatment or diagnosis of Alzheimer's disease and other conditions related to abnormal amyloid beta aggregation.

### BACKGROUND OF THE INVENTION

Alzheimer's disease, for which there is currently limited treatment, constitutes a global public health problem of enormous dimensions. The disease is characterized by progressive dementia that is associated with accumulation of neurofibrillary tangles and amyloid plaques, the latter containing amyloid beta (Aβ), an amphipathic peptide comprising 39-43 amino acids derived by proteolysis from a membrane protein precursor, amyloid precursor protein (APP) (for reviews, see, Lee, V. M., et al., Annu. Rev. Neurosci., 24:1121-1159 (2001), Klein, W. L., Molecular Mechanisms of Neurodegradative Diseases, Chesselet, M. F., Ed., (2000) pp 1-49, Humana Press, Inc. Totowa, N.J.).

Self-association of Aβ is required for toxicity toward neurons in cell culture (Pike, C. J., et al., Brain Res. 563: 311-314 (1991), Lorenzo, A. and Yankner, B. A., Proc. Natl. Acad. Sci. U.S.A. 91: 12243-12247 (1994), Howlett, D. R., et al., Neurodegeneration 4: 23-32 (1995)). Initially, the fibril form was believed to be the toxic species. However, doses of fibrillar Aβ needed to kill neurons in culture appeared excessive (Seubert, P., et al., Nature (London) 359: 325-327 (1992)). Subsequent studies have shown that neurological dysfunction and degeneration can be attributed to smaller, soluble assemblies of Aβ, which have been referred to as soluble oligomers (amyloid-derived diffusible ligands, ADDLs) (Lambert, M. P., et al., Proc.Natl. Acad. Sci. U.S.A. 95: 6448-6453 (1998), Hartley, D. M., et al., J. Neurosci. 19: 8876-8884 (1999), Walsh, D. M., et al., J. Biol. Chem. 274: 25945-25952 (1999)). In particular, a selective neuronal degeneration induced by soluble oligomers has been demonstrated (Kim, H.-J., et al., Faseb J. 17(1): 118-20 (2003)).

Applicants herein have developed a method for the preparation of soluble oligomers in high yield and conditions which stabilize said soluble oligomers.

### SUMMARY OF THE INVENTION

The present invention is a method for producing a stable and soluble preparation of an Aβ oligomer and a composition and formulation thereof. The method uses high concentrations of Aβ peptide, a pH in excess of 7.5 and multivalent anions, such as a buffer with divalent anions, to promote the formulation of Aβ oligomers. In a further embodiment, the method also utilizes additional additives, such as trifluoroethanol and glycerol to enhance the oligomer stability.

In another embodiment of the invention, the product of said method is a stable, soluble Aβ oligomer having a particle size of 10 nm to 100 nm as measured by a dynamic light scattering technique and a molecular weight (Mw) of 100 kDa to 500 kDa.

In a still further embodiment of the invention, the stable, soluble Aβ oligomer is a peptide preparation having at least 50% in the form of oligomers having a diameter of 10 nm to 50 nm and with a Mw of 100 kDa to 500 kDa.

In yet another embodiment of the invention, said peptide preparation is used generate a therapeutic antibody for the treatment of Alzheimer's disease.

### BRIEF DESCRIPTION OF THE DRAWINGS

FIG. 1A represents the effect of pH on soluble Aβ oligomer formation. The Aβ samples were prepared in a sodium buffer adjusted to various pH values between 4.5 and 9.0. FIG. 1B shows the hydrodynamic diameter (Dₕ) distribution of Aβ oligomers obtained from dynamic light scattering analysis with (⋄) representing the mass fraction and (▪) representing the scattering intensity fraction.

FIG. 2 represents the effect of multivalent ions on the formation of soluble Aβ oligomers.

FIG. 3 represents the effect of Aβ peptide concentration on the formation of soluble Aβ oligomers.

FIG. 4 represents the recovery of soluble Aβ oligomer preparations from a HP-SEC column after day 1 and day 4 of storage at 4° C. The total Aβ peak area was integrated and plotted against nominal concentration.

FIG. 5 represents the effect of temperature and various excipients on soluble Aβ oligomer formation. FIG. 5A shows the effects of the various excipients at 37° C., while FIG. 5B shows the effects for the same excipients at 4° C.

FIG. 6 represents the effect of glycerol on soluble Aβ oligomer stability in a sodium phosphate buffer.

FIG. 7 represents the cross-linking of Aβ₄₂ and Aβ₄₀ monomer peptides done with glutaraldehyde. Molecular weight markers are shown on the left as an estimate of size distribution. Lanes 1-5 contains Aβ₄₂, 0%, 0.01%, 0.05%, 0.10% and 0.50% glutaraldehyde, respectively. Lane 6-10 contains Aβ₄₀, 0%, 0.01%, 0.05%, 0.10% and 0.50% glutaraldehyde, respectively.

FIG. 8 represents the stability of soluble Aβ₄₂ oligomers formed in 50 mM phosphate, pH 9.0 buffer, at days 1, 4, and 7 of storage at 4° C. (2-8° C.) as determined by the SDS-PAGE analysis of glutaraldehyde cross-linked samples and non-cross-linked controls. Molecular weight markers are shown as an estimate of size distribution.

FIG. 8A: Lane 1, blank; lanes 2-4, 1 mM stock in 50 mM phosphate, 0.5% glutaraldehyde, days 1, 4 and 7, respectively; lanes 5-7, 1 mM stock, 0% glutaraldehyde, days 1, 4 and 7, respectively; lane 8, MWM, 0.5% glutaraldehyde; lane 9, MWM, 0% glutaraldehyde; lanes 10-12, 850 μM stock, 0% glutaraldehyde, days 1, 4 and 7 respectively.

FIG. 8B: Lane 1, blank; lanes 2-4, 850 μM stock in 50 mM phosphate, 0.5% glutaraldehyde, days 1, 4 and 7, respectively; lane 5, MWM, 0.5% glutaraldehyde; lane 6, MWM, 0% glutaraldehyde; lanes 7-10, 650 μM stock, 0.5% glutaraldehyde, days 1, 4 and 7, respectively; lanes 10-12, 650 μM stock, 0% glutaraldehyde, days 1, 4 and 7, respectively.

FIG. 8C: Lane 1, blank; lanes 2-4, 450 μM stock in 50 mM phosphate, 0.5% glutaraldehyde, days 1, 4 and 7, respectively; lanes 5-7, 450 μM stock, 0% glutaraldehyde, days 1, 4 and 7, respectively; lane 8, MWM, 0.5% glutaraldehyde; lane 9, MWM, 0% glutaraldehyde; lanes 10-12, 250 μM stock, 0% glutaraldehyde, days 1, 4 and 7 respectively.

FIG. 8D: Lane 1, blank; lanes 2-4, 250 μM stock in 50 mM phosphate, 0.5% glutaraldehyde, days 1, 4 and 7, respectively; lane 5, MWM, 0.5% glutaraldehyde; lane 6, MWM, 0% glutaraldehyde; lanes 7-10, 100 μM stock, 0.5% glutaraldehyde, days 1, 4 and 7, respectively; lanes 10-12, 100 μM stock, 0% glutaraldehyde, days 1, 4 and 7, respectively.

FIG. 9 represents the effect of Aβ₄₂ stock concentration in 50 mM phosphate, pH 9.0 buffer, at 4° C. for seven days on in vitro bioactivity in PC-12 cells. Filled squares—5 micromolar test concentration, open circles—1 micromolar test concentration

### DETAILED DESCRIPTION OF THE INVENTION

The standard procedure for the preparation of soluble Aβ oligomers (“Standard Protocol”) utilizes an overnight incubation of Aβ peptide at a concentration up to 100 μM at 4° C. in F12 media (pH 7.4) (Lambert, M. P., et al., Proc.Natl. Acad. Sci. U.S.A. 95:6448-6453 (1998), Chromy B. A., et al., Biochemistry 42: 12749-12760 (2003), Stine W. B., et al., J. Biol. Chem. 278: 11612-11622 (2003)). These studies consistently demonstrated that soluble Aβ oligomer preparations formed under these conditions appeared to contain a mixture of trimers, tetramers (12 kDa-17 kDa) and some larger oligomers in the molecular weight (Mw) range of 50 kDa-200 kDa when analyzed by gel electrophoresis and have a particle size of 3.5 to 10 nanometers in diameter when analyzed by atomic force microscopy (AFM). The Standard Protocol results in a soluble Aβ oligomer preparation in which a high proportion of the mixture is still present as the monomer form. As such, this preparation when used as an antigen has a lower propensity to produce immune response and one in which it is more difficult to recover antibodies specific to soluble Aβ oligomers.

Amyloid beta fibril formation is a complex process that may involve the presence of a transient helical intermediate before the final beta-pleaded conformation is achieved, (Walsh. D. M., et al., J. Biol. Chem. 274: 25945-25952 (1999)). In vitro studies indicate that low concentrations of a helix-inducing solvent, trifluoroethanol (TFE) induces fibril formation at pH 7.4, well below the critical concentration for Aβ fibril formation (at approximately 20 μM). At TFE concentrations above 20%, the helical structure becomes dominant, leading to inhibition of fibril elongation (Fezoui, Y., and Teplow, D. B., J. Biol. Chem. 277: 36948-36954 (2002)). While not wishing to be bound by any theory, Applicants believe that inasmuch as the ionization state of the histidine residues of Aβ affects the association phenomena, raising the pH well above the ionization range would provide conditions where fibril formation is inhibited and where the addition of small amounts of a helix-inducing solvent would promote structure formation and subsequent association. Thus, such conditions would enhance yield and stability of soluble Aβ oligomers. As shown in the examples that follow, Applicants have produced such a stable, soluble Aβ oligomer.

As used herein, the term “soluble Aβ oligomer” means the soluble, oligomeric form of an Aβ peptide. In a preferred embodiment, the soluble Aβ oligomer is the oligomeric form of Aβ₄₂, however, those skilled in the art would recognize that other forms of Aβ, including those containing alterations and mutations could be employed as well. For example, the form of Aβ resulting from the use of a synthetic peptide having mutations at amino acid residues 1 and 2 of the native sequence could be used herein. See, WO 02/094985 and WO 04/099376 for examples of peptides having modifications at amino acid residues 1 and 2 of the native Aβ sequence, incorporated herein as if set forth at length. Another example of a suitable peptide includes the use of a biotinylated form of the Aβ peptide.

As used herein the term “stable, soluble Aβ oligomer” means the soluble, oligomeric form of an Aβ peptide produced by the method claimed herein. By “stable” it is meant a preparation having less monomer relative to the oligomer and one in which the soluble Aβ oligomer so formed is substantially less prone to further associate to form fibrils or aggregates and is less prone to dissociation to form monomers. Using the Standard Protocol known in the art prior to the invention herein, oligomer concentrations up to about 100 μg/ml had a stability of about one day. Following the methods described herein, the oligomers of the present invention having concentrations of 1 mg/ml and higher can be stored for a week at 4° C. The degree of aggregation, used as a measure of stability, was measured using size exclusion chromatography (SEC) techniques by specifically determining the presence or absence of poor peak positions and poor recovery due to the retention of aggregates on pre-filters.

In the present invention Applicants have employed non-standard conditions relative to the Standard Protocol including, increased concentration (more than 100 μM), elevated pH (pH>7.5) and the use of divalent anions to induce the formation of stable, soluble Aβ oligomers. Applicants' improved method produced predominantly stable, soluble Aβ oligomers that are about 10 nm to 50 nm in diameter, as measured by dynamic light scattering, and about 100 kDa to 500 kDa in molecular weight, when measured by static light scattering. In a preferred embodiment, Applicants found the oligomers claimed herein to be 18 nm in diameter and had a measured molecular weight (Mw) of about 155,000 Da. These measurements were confirmed by independently cross-linking and analyzing the resultant oligomers by SDS-PAGE. Applicants believe that previous literature reports underestimate the size of these oligomers, due to the formation of trimers and tetramers in SDS solutions as well as the omission of mobile fragments of polypeptide chains by the scanning probe tip during atomic microscopy measurement. For example, Chromy et al., Biochemistry 42: 12749-12760 (2003) reports the diameter of less than 10 nm based on atomic force microscopy (AFM) and an association state of mostly trimers and tetramers as determined from SDS-PAGE experiments.

The stable, soluble Aβ oligomers of the present invention are suitable for use as an antigen due to their high yield and stability. Said oligomers are particularly stable in the presence of low concentrations of a helix-inducing solvent, such as a 5% solution of TFE. Other organic solvents such as methylene chloride might have helix-inducing properties and can be used for oligomer formation. Propensity to induce helical structure can be individually tested by titrating unstructured peptides in a circular dichroism instrument. Some organic solvents, such as dimethyl sulfoxide, that do not have helix-inducing properties are well suited for preparation in initial monomer stock solutions.

Moreover, inasmuch as Aβ is a self-antigen, it would be advantageous to create an oligomer that has a structure similar to naturally occurring toxic diffusible oligomers and that is highly immunogenic in order to break immune tolerance. Those of ordinary skill in the art know that antigens that associate into large assemblies are generally more immunogenic (see, for example, Kovacsovics-Bankowski, M., et al., Proc. Natl. Acad. Sci. USA 90: 4942-4946 (1993)). The availability of structurally relevant, stable, soluble Aβ oligomers would be of benefit in the generation, selection and quality control of therapeutic monoclonal antibodies. As such, the stable, soluble Aβ oligomers of the present invention would provide an improved preparation in the development of an antigen for a passive immunization approach to the treatment of AD and other diseases associated with abnormal Aβ aggregation.

One embodiment of the present invention comprises a stable, soluble Aβ oligomer that is 10 nm to 50 nm in diameter and represents a homogenous population that is dominant in the sample. In a preferred embodiment the soluble Aβ oligomer of the present invention comprises at least 50% of the peptide antigen preparation, when formed at concentrations higher than 100 μM, at pH 7.5 or higher, and in the presence of divalent anions. More preferably, the stable, soluble Aβ oligomer comprises at least 70% of the peptide antigen preparation and, most preferably, the stable, soluble Aβ oligomer of the instant invention comprises at least 90% of the peptide antigen preparation.

It should be noted that the apparent size of soluble Aβ oligomer may differ from that determined by dynamic light scattering when using an atomic force microscopy (AFM) technique in which solid matter is detected by a probe tip. In such instances, the resulting size determinations may be an underestimation of the actual oligomer size due to presumed inability of the tip to register peptide ends that are loosely suspended in the solution. In contrast, when using a dynamic light scattering technique, these loosely suspended ends provide a substantial contribution to the overall diffusion coefficient and tend to increase the resulting hydrodynamic size (Koppel, D. E., J. Chem. Phys. 37:4814-4820 (1972)). The presence of an unstructured outer layer of the oligomer is consistent with lack of structure reported for the N-terminus of the peptide in fibrils (Petkova et al., Proc. Nat. Acad. Sci. USA 99: 16742-16747 (2002)).

Without wishing to be bound by any theory, Applicants believe that the properties of the stable, soluble Aβ oligomers described herein result, in part, from the use of relatively high pH in its preparation and storage. The Aβ peptide is composed of six negatively charge amino acid residues (three aspartic acid residues and three glutamic acid residues) and six potentially positive amino acid residues (one arginine, one lysine residue, one terminal amino group and three histidine residues). The presence of three histidine residues that have a nominal ionization constant pKi at pH 6.5, will tend to ionize (become positive) at acidic and neutral pH, while remaining neutral (deprotonated) at high pH. Ionization of the three histidine residues results in neutralization of the net peptide charge and accelerated association due to lack of charge repulsion. In contrast, deprotonation of the histidine residues results in an overall net of three negative charges which will make association more selective. As a result, most of the published protocols for the formation of fibrils call for the use of low pH and low ionic strength, conditions that will maximize electrostatic interactions. In this way, those of ordinary skill in the art would recognize that Applicants' use of an elevated pH in the instant method to form stable, soluble Aβ oligomers differs from the teachings of known methods of preparing fibrils.

The stable, soluble Aβ oligomers described herein are preferably formed and stored in the presence of multivalent anions. Again, without wishing to be bound by any theory, Applicants believe that this preference may be related to the known affinity Aβ has for lipid membranes that contain phosphatidylinositol, a negatively charged lipid that contains a phosphate group. The preference for the presence of multivalent anions may also be related to the affinity Aβ has for monosialoganglioside (GM1). It is known that GM1 assembles into micelles in aqueous solutions to form an oligosaccharide surface that contains negative charged carboxylic groups. Typically, phosphate ions would be the multivalent anion of choice. However, due to the known covalent binding of phosphate ions to aluminum hydroxide-containing adjuvants, such as Merck aluminum adjuvant (Klein et al., J. Pharm. Sci. 89: 311-321 (2000)), the use of sulphate ions is preferred when an aluminum hydroxide-containing adjuvant is to be used as part of the antigen preparation.

The amphipathic properties of Aβ are apparent from its ability to partition into membranes containing phosphatidylinositol or into GM1 micelles. Despite increasing concentrations of the peptide, in the absence of TFE, it appears that about 100 μM concentration of the peptide remains in the monomeric form. Such an observation is consistent with surfactant-like properties reported for the peptide (Kim, J. and Lee, M., Biochem. Biophys. Res. Commun. 316(2): 393-7 (2004)) and, as such, this property has been used by Applicants to achieve high yields of the stable, soluble Aβ oligomers by increasing the concentration to 200 μM and higher. In contrast, prior attempts to form oligomers by using longer reaction times (7 days) at a 100 μM concentration and at a physiological pH (7.4) resulted only in the formation of an excess population of the fibrils (Stine, W. B., et. al, J. Biol. Chem. 278:11612-11622 (2003)).

Applicants have also found that the amphipathic property of Aβ and its surfactant-like behavior is also demonstrated in the stable, soluble Aβ oligomers of the invention upon their dilution with a solvent that has dielectric constant significantly lower than water, such as glycerol. This aspect of the invention may be useful for experiments involving ligand screening when the original oligomer sample needs to be applied under conditions of lower concentrations and dissociation of the particles is to be minimized. The presence of glycerol would result in higher proportion of oligomers remaining in original oligomerization state after dilution and thus would presumably lead to higher avidity in binding assays or experiments.

The temperature used in the preparation of the stable, soluble Aβ oligomers is also believed to be important, as elevated temperatures are known to accelerate aggregation (Stine et al., J. Biol. Chem. 278: 11612-11622 (2003)). Applicants have found that temperatures in the range of 2° C. to 8° C. are to be employed so as to minimize the formation of fibrils.

In one embodiment of the invention, the preparation of the stable, Aβ oligomers employs the use of helix-inducing organic solvents at 37° C. to accelerate oligomer formation and stabilize the oligomers in storage by minimizing fibril formation. In such an embodiment, the method uses TFE to promote the conversion of the monomeric peptide into the soluble oligomers and to stabilize the soluble oligomers. This method of formation of the stable, soluble Aβ oligomers is preferred when the toxicity of TFE is not relevant or it can be removed, for example, by a settle-decant approach after binding to an aluminum adjuvant. In the absence of such a stabilizing solvent, the use of a low temperature (2° C. to 8° C.), in addition to relatively high pH and concentration, is needed to achieve optimal stability (minimum 7 days).

Further, inasmuch as it appears that the stability of the soluble Aβ oligomers herein are dependent on concentration, chemical cross-linking may protect the oligomers so produced from decomposition resulting from dilution. Thus, in one embodiment of the invention glutaraldehyde is used to protect the oligomers from decomposition, as tested with SDS treatment.

### EXAMPLES

### Example 1

### Effect of pH on Formation and Size of Soluble Aβ Oligomers

All chemicals and reagents were obtained from Sigma-Aldrich (St. Louis, Mo.) unless otherwise noted.

The Aβ peptide (1-42) (Aβ₄₂) (American Peptide, Sunnyvale, Calif.) was dissolved in 100% hexafluoroisopropanol (HFIP), distributed into 2 mg aliquots into 1.7 ml polypropylene tubes and subjected to centrifugation under vacuum and low temperature (CentriVap Concentrator, Labconco, Kansas City, Mo.) until the solvent was evaporated. Dry films were protected from moisture and stored at −70° C. until use. The peptide stock solution was prepared by adding 100 μL anhydrous dimethyl sulfoxide (DMSO) to 2 mg dry film after equilibration in room temperature and gently mixed by repetitive aspiration with a pipette. Stock solutions were stored at room temperature for up to 2 weeks.

The Aβ samples (100 μM) were prepared in 50 mM sodium phosphate buffer adjusted to various pH values between 4.5 and 9.0 and incubated at 4° C. for 3 days. The samples were centrifuged at 7,000 rpm for 3 minutes on a table top centrifuge (7 cm radius) to remove large aggregates or fibrils and then filtered through 0.22 micron filters (Millipore, Bedford, Mass.) to remove particles that are too large for the size-exclusion column. Ten μl of each filtrate was injected onto size-exclusion chromatography (SEC) column. The stable, soluble Aβ oligomers' peak eluted at approximately 6.5 ml, while the peak for the monomer eluted at approximately 9 ml.

Size exclusion chromatography was performed using an Alliance® HPLC System (Waters Corporation, Milford, Mass.) employing a Waters® Protein PAK 125 7.8×300 mm column. The running buffer was 50 mM sodium phosphate, pH 9 eluted at 1 ml/min. The minimum amount of injected peptide was 25 μg. The photo-diode-array UV detector was set for detection between 210 and 350 nm with 3.5 nm resolution. The spectra of oligomer and monomer peaks were occasionally examined to confirm the identity of the peaks. The complete UV readout was transferred into a spreadsheet format (Excel, Microsoft Corporation, Redmond, Wash.) where UV absorbance at 230 nm was extracted and plotted against elution volume. In some instances the area under the peaks was integrated using build-in functions and the oligomer fraction (i.e., the fraction of total material eluted between 5 ml and 7.5 ml), as well as total recovery, was estimated.

Static and dynamic light scattering analysis were performed to determine oligomer size using a Malvern 4700 system (Malvern Instruments, Southborough, Mass.) equipped with 1 W 488 nm Argon laser, following centrifugation at 40,000 r.p.m. in a rotor of approximately 4.5 cm radius for 15 minutes (Beckman Optima ultracentrifuge) to remove a small (<5%) fraction of aggregates that were about 200 nm in diameter. Typically, results from five measurements, each done for three minutes, were averaged. Data was analyzed using a nonlinear least-squares fitting procedure (Malvern Instruments).

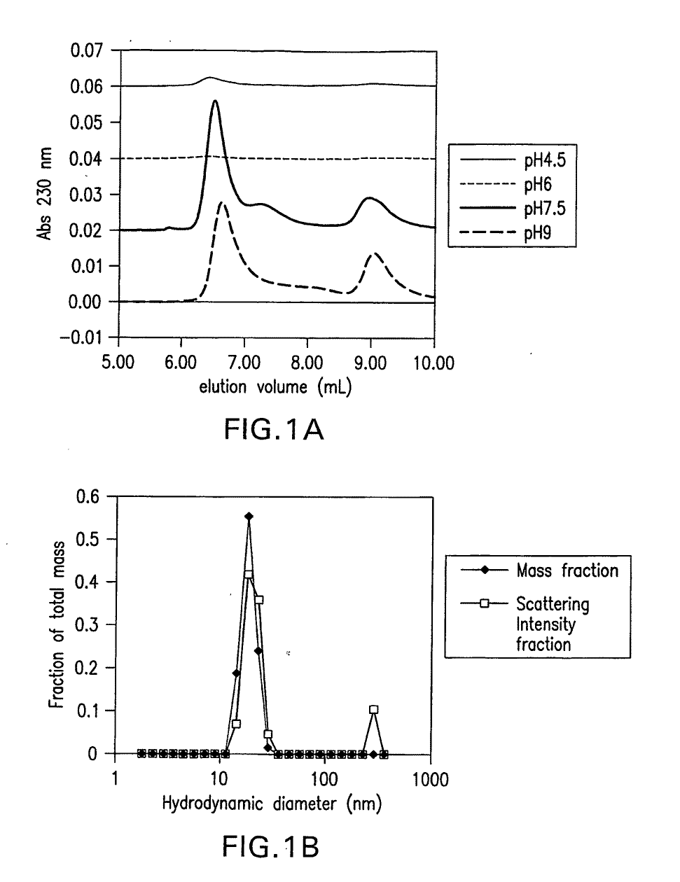

The results of high-pressure size exclusion chromatography (HP-SEC) analysis of soluble oligomer samples prepared at various pH levels are shown in FIG. 1A. The area under the peaks for samples incubated at pH 4.5 and pH 6 indicates that a substantial loss of the initial material occurred. The sample incubated at pH 6 was visibly turbid and most of the sample was removed upon gentle centrifugation and filtration through a 0.22 micron (220 nm) filter. The sample incubated at pH 4.5 appeared clear, but substantial fibril/aggregate formation occurred, since the majority of the mass was lost in the centrifugation and filtration steps. Both samples that were incubated at a pH above 7.0 showed complete recovery and majority of the mass present in the form of oligomer. In preferred embodiments of the invention, the pH is maintained at a level above 8 to provide control of the rate of oligomerization. Use of a pH 7.4, 50 mM sodium phosphate, buffer at 2° C. to 8° C. to form and store oligomers resulted in lower storage stability, as judged by higher proportion of the material that further associated and did not elute from HP-SEC column (not illustrated).

In order to determine the size of the Aβ oligomers so formed, Applicants subjected the samples to a dynamic light scattering analysis. A non-linear least squares (NLLS) analysis indicated that the majority of the mass existed as particles of about 20 nm in diameter and that a small amount (less than 5%) existed as very large particles (about 200 nm in diameter). Since the intensity of scattered light is proportional to the molecular weight of the scattering particles, and the larger particles, which were estimated to have Mw in excess of 1 million Daltons contributed about 50% of total light scattering intensity, Applicants used a centrifugation step to remove larger particles. Centrifugation at 40,000 rpm for 15 minutes in a rotor of 4.5 cm radius was sufficient to remove most of the large particles (about 200 nm). Total mass loss in this centrifugation step was about 3% as judged by UV absorbance at 275 nm (data not shown).

The results of the light scattering analysis of the centrifuged Aβ oligomer sample prepared at pH 9 and measured at 450 μM is presented in FIG. 1B. The analysis of the fluctuations of the scattered light allows the determination of the diffusion coefficients and, consequently, the hydrodynamic diameter distribution. The diameter of the major soluble Aβ oligomer was found to be 18.9 nm+/−0.3 nm using non-linear least-squares fitting. Essentially the same results of Dₕ=21.4 nm+/−0.7 nm were previously obtained for oligomers formed using the Standard Protocol (data not shown).

### Example 2

### Effect of Divalent Anions on the Formation of Soluble Aβ Oligomers

This example shows the effect of buffering component valency on the formation of Aβ oligomers. The buffers were prepared at 50 mM concentration and the pH was adjusted to 9.0 using 1M hydrochloric acid or sodium hydroxide. 220 μM samples were incubated overnight at 4° C. and analyzed by HP-SEC. Peaks between 6 and 8 minutes and between 8 and 9.5 minutes were integrated to yield peak areas of the oligomer and monomer, respectively. Sodium was used as a cation in all cases.

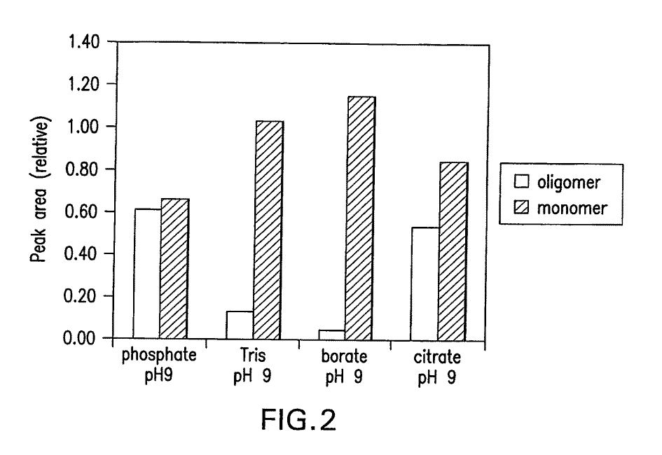

The results of the HP-SEC analysis of a 220 μM preparation of soluble Aβ oligomers that were incubated at 4° C. overnight are shown in FIG. 2. The preparation that contained multivalent anions (phosphate and citrate) showed a significantly greater proportion of soluble Aβ oligomers than those prepared in the presence of monovalent ions (Tris and borate).

### Example 3

### Effect of Aβ Concentration on the Formation of Soluble Aβ Oligomers

This example shows the effect of Aβ concentration on the formation of soluble Aβ oligomers. Aβ 20 mg/ml stock solution in 100% DMSO were dissolved in 50 mM sodium phosphate at various proportions and incubated overnight at 4° C. A HP-SEC analysis was performed and the total area of the soluble Aβ oligomer peak divided by the total area of the sum of monomer and oligomer peaks. High concentration samples were also tested after an additional 3 days of incubation at 4° C.

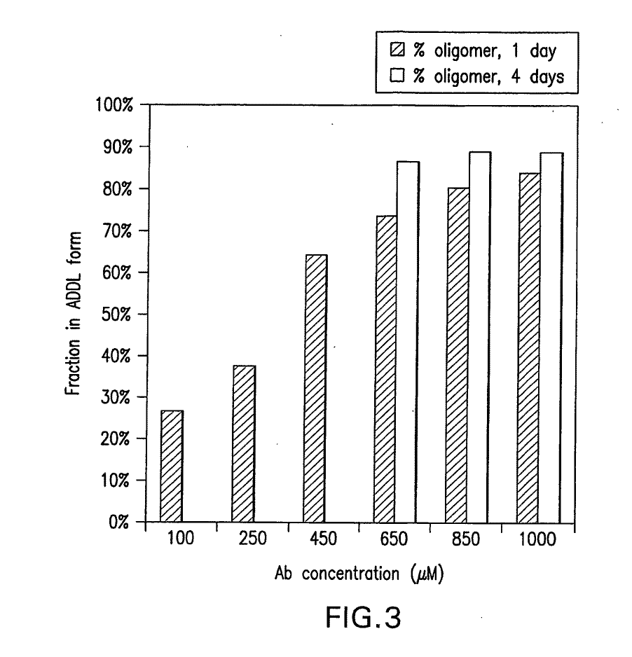

FIG. 3 shows that increasing the concentration of Aβ leads to an increased proportion of soluble Aβ oligomers. For high concentration samples the process of formation is close to completion after an overnight incubation. Furthermore, the effect of increased concentration appears to saturate when about 90% of the peptide is converted to the oligomeric species. This suggests that there is a solubility limit of the peptide, similar to critical micellar concentration (cmc) of surfactants.

### Example 4

### Stability of Soluble Aβ Oligomers

This example shows the recovery of soluble Aβ oligomers from a HP-SEC column after 1 day and 4 days of storage at 4° C. The total Aβ peak area was integrated and plotted against nominal concentration.

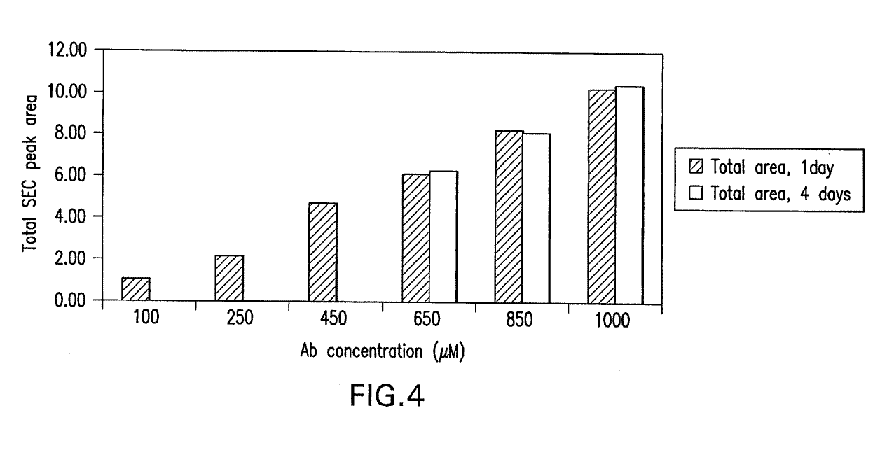

The experiment described in Example 3 was also used to estimate the stability of preparations upon incubation at 4° C. FIG. 4 shows total peak area at various concentrations after overnight and 4 days incubation. The total amount of material recovered from the HP-SEC column as judged by UV detection did not change between 1 day and 4 days, indicating that these preparations are stable in storage.

### Example 5

### Effect of Temperature and Excipients on the Formation and Stability of Soluble Aβ Oligomers

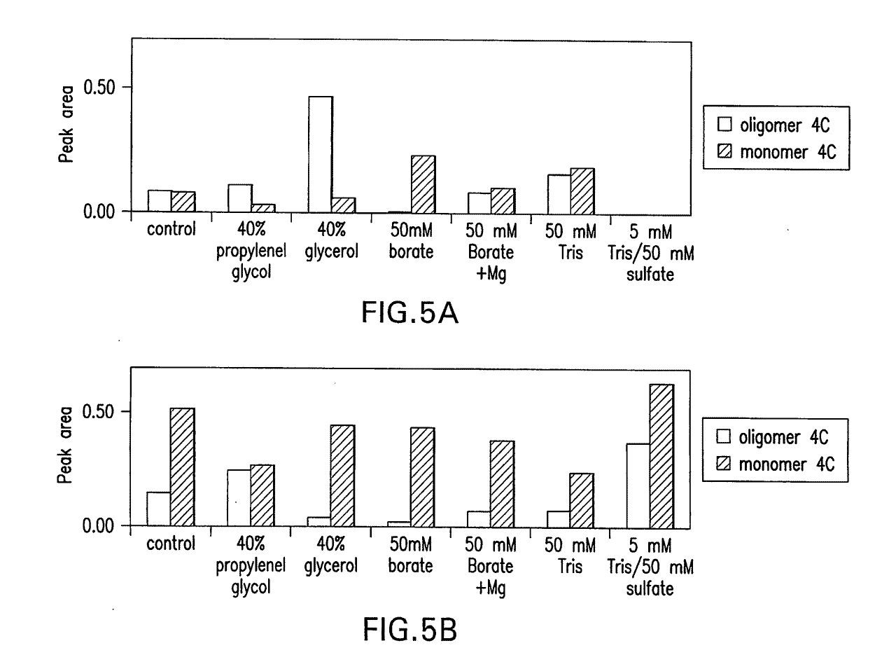

This example shows the effect of temperature and excipients on soluble Aβ oligomer formation. The samples were prepared at 100 μM concentration and tested by HP-SEC followed by peak integration. The samples were evaluated at 37° C. (FIG. 5A) and 4° C. (FIG. 5B). The buffer was 50 mM sodium phosphate unless otherwise noted.

As seen in FIG. 5A, poor recovery was observed in the samples incubated at 37° C., except for the sample containing 40% glycerol. Examination of the proportions of soluble Aβ oligomers from the corresponding 4° C. sample (FIG. 5B) showed that the presence of glycerol inhibited the formation of soluble Aβ oligomers (compare to control). Without wishing to be bound by any theory, this inhibition may form the basis for the stabilizing effect observed when glycerol was added after oligomers already formed. In addition, the presence of divalent anions (phosphate and sulfate), as well as propylene glycol, appeared to promote soluble Aβ oligomer formation. It is also appears that formation of soluble Aβ oligomers at 4° C. occurred in a practically applicable kinetic scale, consistent with the stability observed in the previous experiments (Example 3 and 4). Further, since it was noted that soluble Aβ oligomers partially dissociate upon dilution, by comparison to surfactant properties, an inert excipient that has much lower dielectric constant was tested to establish its effect on the stability of soluble Aβ oligomers. As seen in FIGS. 5A, 40% glycerol exhibits such a stabilizing effect at 37 deg C.

### Example 6

### Effect of Glycerol on the Stability of Soluble Aβ Oligomers in Dilute Solutions

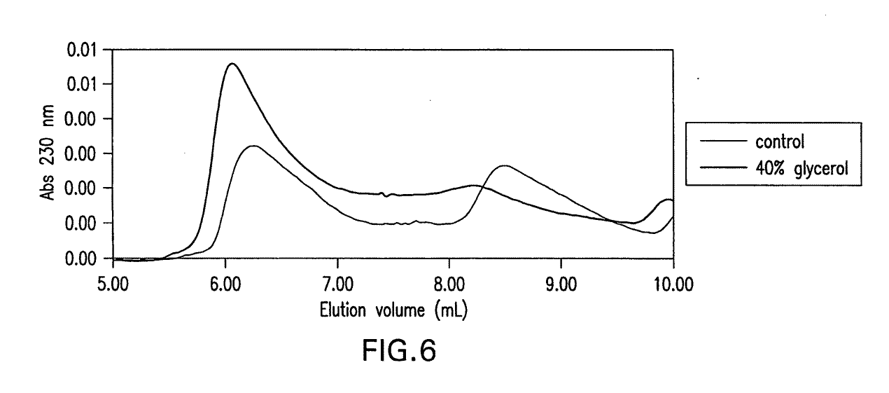

This example shows the inhibition of dissociation of soluble Aβ oligomers prepared at 440 μM concentration and diluted four fold into 50 mM sodium phosphate buffer, pH 9, in the presence of 40% glycerol (FIG. 6). An identical sample, but formulated without glycerol, served as a control. Samples were incubated overnight at 4° C. before injecting them onto a HP-SEC column. A higher proportion of oligomers were observed in the sample containing glycerol.

### Example 7

### Stabilization of Soluble Aβ Oligomers by Chemical Cross-Linking

This example shows the concentration of glutaraldehyde necessary to cross-link soluble Aβ oligomers. One of the potential problems associated with oligomers formed under optimal conditions is that they dissociate upon dilution. On the other hand, extensive chemical modification usually leads to the loss of bioactivity. This example shows the optimal concentrations of the cross-linking agent for cross-linking the oligomer for analytical purposes (relatively high concentration of glutardehyde with loss of bioactivity) and for preparation of the material to be used in biological experiments (relatively low concentration of glutaraldehyde with preservation of bioactivity)

The soluble Aβ oligomers were cross-linked with glutaraldehyde, incubated for 10 minutes at room temperature and then quenched with 1M glycine, 1M Tris-HCl pH 7.5. Cross-linked samples were then diluted to a final concentration of 0.02 μg/μL, in Tris-glycine SDS sample buffer. For analysis, 0.28 μg Aβ (nominal concentration) in either monomeric or oligomeric form was separated by electrophoresis at 125V for about 100 minutes using a 4% to 20% tris-glycine gel (Invitrogen, Carlsbad, Calif.). Gels were then silver stained to visualize the size distribution of the soluble Aβ oligomers. For glutaraldehyde concentration optimization, HFIP-dried Aβ₄₂ or HFIP-dried Aβ₄₀ was solubilized in DMSO (20 mg/mL, 4.4 mM), added to 50 mM phosphate, pH 9.0, to a final concentration of 1.8 mg/mL (400 μM) and incubated at 4° C. overnight, protected from light. Following the overnight incubation, 36 ng of the Aβ protein was cross-linked with various concentrations of glutaraldehyde ranging from 0 to 0.5%.

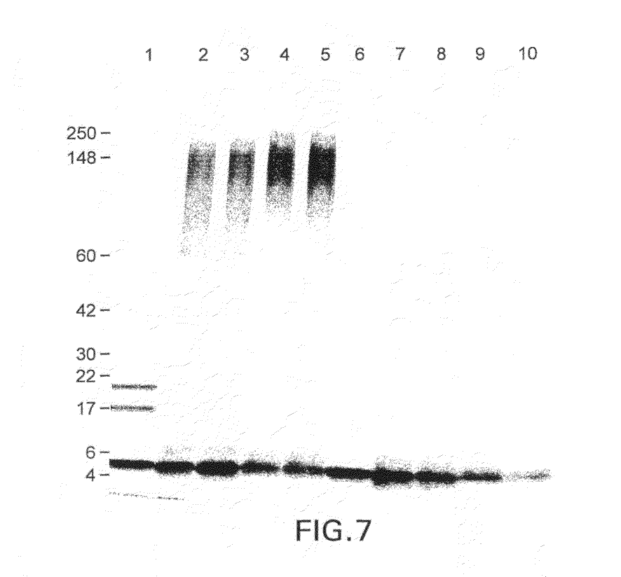

The silver stained gel shown in FIG. 7 below is representative of the data obtained from this experiment. Results indicate that 0.1% glutaraldehyde is sufficient to cross-link soluble β₄₂ oligomers that have formed during a 4° C. overnight incubation. At concentrations of 0.01 and 0.05% glutaraldehyde, a small amount of dimer (8 kDa) is visible in lanes 2 and 3. Lanes 5 through 10 indicate that Aβ₄₀ primarily stays monomeric and does not form oligomers during a 24 hour incubation at 4° C. in 50 mM phosphate, pH 9.0. The bands seen at the 17 kDa range represent the product of the SDS treatment that was not present before SDS addition; they would have been cross-linked if they were originally present in the sample, i.e. before SDS was added. Thus, soluble Aβ oligomers appear to have molecular weight in the range of 150 kDa, which is in contrast to the consensus in the art that indicated a molecular weight in the range of 20 kDa.

As a result of this example, Applicants concluded that this methodology would be effective to monitor formation and determine size distribution of soluble Aβ oligomer preparation.

### Example 8

### Stability of Soluble Aβ Oligomers in 50 mM Phosphate, pH 9.0

As set forth above in Examples 1-4, Aβ₄₂ forms soluble Aβ oligomers in 50 mM phosphate, pH 9.0 buffer during storage at 2° C. to 8° C. However, the optimal concentration for formation, biological activity and storage stability of these oligomeric species during storage was not determined. As described in Example 7, a method was developed in which 0.1% glutaraldehyde is used to cross-link Aβ₄₂ oligomeric species for analytical purposes. These cross-linked species were not disrupted in the presence of SDS, and therefore, the appropriate size distribution could be determined by SDS-PAGE. In this example, Aβ₄₂ was prepared at concentrations of 1 mM, 850 μM, 650 μM, 450 μM, 250 μM and 100 μM in 50 mM phosphate pH 9.0 buffer as stated above. After 1, 4 and 7 days of incubation at 4° C., the samples were cross-linked with 0.5% glutaraldehyde or an equivalent amount of water to serve as a non-cross-linked control and then separated by SDS-PAGE as described in Example 7.

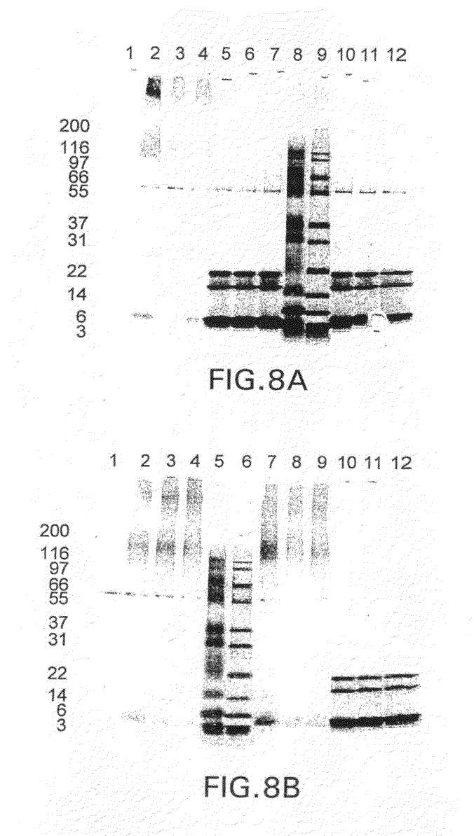

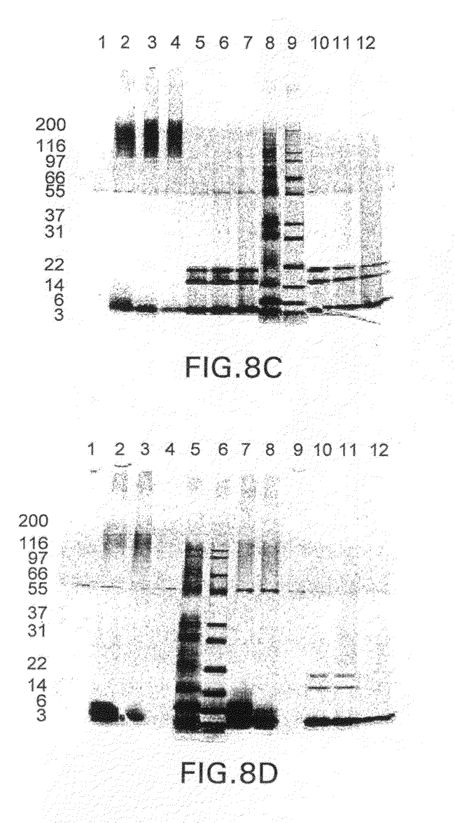

As shown in FIG. 8, at an Aβ₄₂ concentration of 1 mM, soluble Aβ oligomers (at about 150 kDa, hereinafter the “150 kDa species”, equivalent to 35-mers and higher) are formed after one day of incubation at 4° C. as observed in FIG. 8A, lane 2. After 4 and 7 days of storage at 4° C., the amount of this species is reduced and a higher molecular weight material is observed (FIG. 8A, lanes 3 and 4). A similar pattern is observed for the 850 μM and 650 μM Aβ₄₂ stock concentrations (FIG. 8B lanes 2, 3, 4, 7, 8 and 9, respectively), but the relative amount of the 1500 kDa species gradually declines during the storage period. However, with an Aβ₄₂ stock concentration of 450 μM, the amount of the 150 kDa species remains consistent during seven days of storage at 4° C. and little to no higher molecular species are observed (FIG. 8C, lanes 2, 3 and 4). Finally, Aβ₄₂ stock concentrations of 250 μM and 100 μM result in species that after 1 day of storage at 4° C. remains primarily monomeric (at about 4 kDa) with little of the 150 kDa species being formed (FIG. 8D, lanes 2 and 7). With additional storage time at 4° C., the amount of the 150 kDa species does not increase, while the apparent amount of monomer does appear to be decreasing (FIG. 8D, lanes 3, 4, 8 and 9). This suggests that the Aβ₄₂ peptide is degrading over time. Based on these results, Applicants concluded that Aβ₄₂ at stock concentration of 450 μM forms a relatively stable oligomeric species in 50 mM phosphate, pH 9.0 buffer that can be stored for seven days at 4° C. This data, in addition to the MTT assay data presented below in Example 9, supports the use of a 450 μM Aβ₄₂ stock in 50 mM phosphate, pH 9.0 buffer as a relatively stable, bioactive soluble Aβ oligomer.

### Example 9

### Assessment of Toxicity of Soluble Aβ Oligomers

It has been previously shown (Examples 1-4) by HP-SEC that Aβ₄₂ forms soluble Aβ oligomers in 50 mM phosphate, pH 9.0 buffer. These soluble Aβ oligomers were also shown to be bioactive in the PC-12 MTT (3-(4,5-dimethyl-2-thiazolyl)-2,5-diphenyl-2H-tetrazolium bromide) reduction assay (Example 10). Yet the critical concentration of Aβ₄₂ to form these species and/or the critical concentration that provides maximum cellular bioactivity was unknown. Therefore, a study was conducted to determine the critical concentration of the Aβ₄₂ stock that fits both of these conditions.

PC-12 cells were plated at 30,000 cells/well and allowed to grow overnight at 37° C./5% CO₂. Soluble Aβ oligomers or vehicle were added to cells at concentrations of 1 μM and 5 μM. After a four hour incubation at 37° C./5% CO₂, the MTT reduction assay was performed (Lambert et al., 2001, J. Neurochem. 79, 595-605). Briefly, MTT (10 μL, 5 mg/mL) was added to each well and allowed to incubate for four hours. A solubilization buffer (100 μL, 10% SDS in 0.01 N HCl) was added and the plate was incubated at 37° C./5% CO₂ overnight. The assay was then quantified at 595 nm on a Tecan Spectrafluor Plus plate reader (Tecan Systems, San Jose, Calif.).

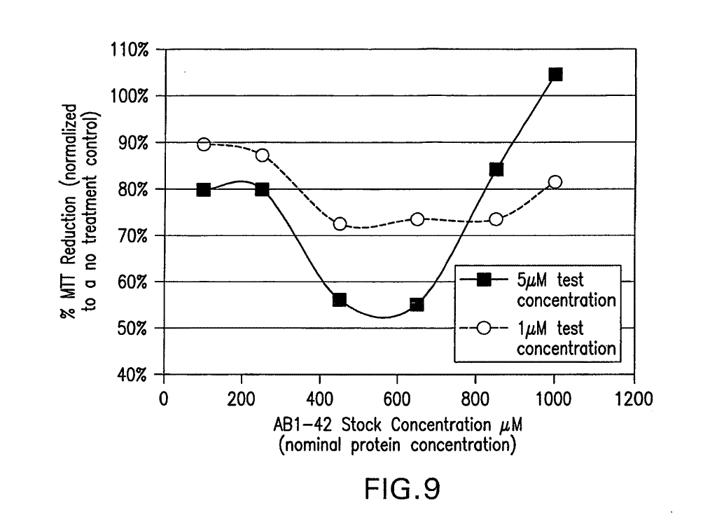

In this experiment, Aβ₄₂ was prepared at concentrations of 1 mM, 850 μM, 650 μM, 450 μM, 250 μM and 100 μM in 50 mM phosphate pH 9.0 buffer as described in the Example 8. After 7 days of incubation at 4° C., samples were tested by the MTT assay to determine bioactivity. Results for these samples tested at nominal concentrations of 1 μM and 5 μM are shown in FIG. 9. Results indicate that Aβ₄₂ is highly bioactive at stock concentrations in the range of 450 μM to 650 μM. At a test concentration of 5 μM, these stock concentrations showed about 55% MTT reduction. In contrast, stock concentrations below 450 μM and above 650 μM were shown to have little bioactivity, displaying 80-105% MTT reduction in PC-12 cells. Based on these results, Applicants concluded that Aβ₄₂ at stock concentrations of 450 μM to 650 μM form soluble Aβ oligomers in 50 mM phosphate, pH 9.0 buffer that remain bioactive for up to seven days of storage at 2-8° C.

### Example 10

### Preparation of Stable, Soluble Aβ Oligomers

The Aβ peptide (1-42) (Aβ₄₂) (American Peptide, Sunnyvale, Calif.) was dissolved in 100% hexafluoroisopropanol (HFIP), distributed into 2 mg aliquots into 1.7 ml polypropylene tubes and subjected to centrifugation under vacuum and low temperature (CentriVap® Concentrator, Labconco, Kansas City, Mo.) until the solvent was evaporated. Dry films were protected from moisture and stored at −70° C. until use. The peptide stock solution was prepared by adding 100 μL anhydrous dimethyl sulfoxide (DMSO) to 2 mg dry film after equilibration in room temperature and gently mixed by repetitive aspiration with a pipette. Stock solutions were stored at room temperature for up to 2 weeks.

The Aβ stock solution is added at various ratios to 50 mM sodium phosphate, pH 9, while vortexing at room temperature to obtain final peptide concentration between 400 and 700 μM. Sample is transferred to 2-8° C. and stored at least one day before use.

### Example 11

### Preparation of Stable, Soluble Aβ Oligomer Antigen

Stable, soluble oligomer prepared as described in Example 8 is prepared, except 50 mM sodium sulfate is used instead of sodium phosphate. Small amount of monovalent buffer (e.g. 10 mM Tris) is added to maintain pH above 8.0. After overnight incubation at 2-8° C., oligomeric sample is added to Merck aluminum adjuvant while mixing on vortex. Final buffer is introduced by centrifuging the sample to pellet alum, exchange of the supernatant and resuspension of antigen-alum complexes on vortex. Optionally, non-alum adjuvants may also be introduced. Optionally, aluminum phosphate or sodium phosphate-prepated oligomers can be used when binding to alum is to be minimized.

### Example 12

Use of Stable, Soluble Aβ Oligomer Antigen Preparation to Generate Antibodies

Antigen-alum complexes are injected into animals, preferably in a repetitive manner. The animals are sacrificed and spleen cells are mixed with myeloma cells and subjected to fusion. These fused hybrid cells are then cultured and the supernatants harvested from these cultures are screened for the presence of anti-oligomer antibodies. Positive clones are multiplied for production of monoclonal antibodies.

Alternatively, Aβ oligomers are immobilized on 96-well plates and phage libraries are screened for the ability to recognize the Aβ oligomeric antigen. Positive phage species are multiplied and used for antibody production.

1. A method for producing a stable, soluble amyloid beta (Aβ) oligomer comprising:
(a) obtaining a concentrated stock solution of Aβ peptide in an organic solvent; and
(b) adding said concentrated stock solution of the peptide to an aqueous solution having at least 10 mM of a divalent anion and buffered to a pH of at least 7.5 to form a reaction mixture having a final peptide concentration in excess of 100 μM;
wherein the stable, soluble Aβ oligomer is formed in the reaction mixture of step (b) upon standing and comprises at least 50% of said reaction mixture.

2. A method of claim 1 further comprising incubating the reaction mixture of step (b) at a temperature of 2° C. to 8° C.

3. A method of claim 1 further comprising separating the reaction mixture of step (b) by size exclusion chromatography to produce eluted oligomeric fractions and recovering said stable, soluble Aβ oligomer from the eluted oligomeric fractions.

4. The method of claim 1 further comprising the addition of a helix inducing organic solvent to the aqueous solution of step (b).

5. The method of claim 1 wherein the stock solution has an Aβ concentration of 200 μM to 900 μM.

6. The method of claim 1 wherein the reaction mixture has a pH of 7.5 to 11.0.

7. The method of claim 1 wherein the divalent anions are selected from the group consisting of phosphate ions and sulphate ions.

8. The method of claim 4 wherein the helix inducing excipient is 2% to 15% trifluoroethanol.

9. A stable, soluble Aβ oligomer produced by the method of claim 1.

10. A stable, soluble Aβ oligomer of claim 9 that is stored in the presence of 5% to 50% glycerol.

11. The oligomer of claim 9 having a particle size of 10 nm to 100 nm in diameter.

12. The oligomer of claim 9 having a molecular weight of 100 kDa to 500 kDa.

13. An isolated, stable, soluble Aβ oligomer preparation wherein said oligomer preparation comprises particles of dimensions of 10 to 100 nm in diameter as measured by a dynamic light scattering technique.

14. The oligomer of claim 13 having a molecular weight of 100 kDa to 500 kDa.

15. A method for producing a stable, soluble amyloid beta (Aβ) oligomer comprising:
(a) obtaining a concentrated stock solution of Aβ peptide in an organic solvent;
(b) adding said concentrated stock solution of the peptide to an aqueous solution having at least 10 mM of a divalent anion and buffered to a pH of at least 7.5 to form a reaction mixture having a final peptide concentration in excess of 100 μM; and
(c) formulating the stable, soluble Aβ oligomer formed in the reaction mixture of step (b) with an adjuvant;
wherein the stable, soluble Aβ oligomer comprises at least 50% of said reaction mixture of step (b).

16. A method of claim 15 further comprising incubating the reaction mixture of step (b) at a temperature of 2° C. to 8° C.

17. A method of claim 15 further comprising separating the reaction mixture of step (b) by size exclusion chromatography to produce eluted oligomeric fractions and recovering said stable, soluble Aβ oligomer from the eluted oligomeric fractions.

18. The method of claim 15 further comprising the addition of a helix inducing organic solvent to the aqueous solution of step (b).

19. A method of claim 18 wherein the helix inducing excipient is 2% to 15% trifluoroethanol.

20. A stable, soluble Aβ oligomer produced by the method of claim 15.

21. A stable, soluble Aβ oligomer of claim 20 that is stored in the presence of 5% to 50% glycerol.

22. The oligomer of claim 20 having a particle size of 10 nm to 100 nm in diameter.

23. The oligomer of claim 20 having a molecular weight of 100 kDa to 500 kDa.
